(19) United States
(12) Patent Application Publication
(10) Pub. No.: US 2025/0380857 A1
(43) Pub. Date: Dec. 18, 2025

(54) OPTICALLY-GUIDED URETERAL STENT

(71) Applicant: ARIZONA BOARD OF REGENTS ON BEHALF OF THE UNIVERSITY OF ARIZONA, Tucson, AZ (US)

(72) Inventors: John HEUSINKVELD, Tucson, AZ (US); Jennifer BARTON, Tucson, AZ (US); Dominique GALVEZ, Tucson, AZ (US); Andrew ROCHA, Tucson, AZ (US); William DRAKE, Tucson, AZ (US)

(21) Appl. No.: 19/107,819

(22) PCT Filed: Aug. 30, 2023
(86) PCT No.: PCT/US2023/031557

§ 371 Date: Feb. 28, 2025

Related U.S. Application Data
(60) Provisional application No. 63/373,935, filed on Aug. 30, 2022.

(51) Int. Cl. A61B 1/005; A61B 1/00; A61B 1/018; A61B 1/05; A61B 1/06; A61B 1/307; A61B 5/00

(52) U.S. Cl. CPC A61B 1/0052; A61B 1/00045; A61B 1/00096; A61B 1/00117; A61B 1/00119; A61B 1/00128; A61B 1/018; A61B 1/05; A61B 1/0676; A61B 1/307; A61B 5/0066; A61B 5/6847

(57) ABSTRACT

An optically-guided ureteral stent includes an elongate body and an imager. The elongate body has a plurality of channels therethrough and a curve near a proximal end of the elongate body. The imager is disposed at the proximal end of the elongate body in a first channel of the plurality of channels. A second channel second channel of the plurality of channels has an opening at one of the proximal end and a lateral surface of the elongate body for passage of urine.

### CROSS-REFERENCE TO RELATED APPLICATIONS

This application claims the benefit of U.S. Provisional Application No. 63/373,935, filed on 30 Aug. 2022, the disclosure of which is incorporated herein by reference in its entirety.

### BACKGROUND

Tubes planted within ureters are known as ureteral stents. Ureteral stents are used to ensure adequate passage of urine when a ureter has been damaged by, for example, removal of a kidney stone from the ureter or passage of a stone through the ureter. They are also used following surgery that damages or repairs ureters to ensure healing tissue does not block the ureter, and when a tumor presses on, and cuts off flow within, the ureter.

Prior art ureteral stents have no optical guidance or inspection systems of their own; prior art ureteral stents are typically inserted into the ureteral opening of the bladder through a cystoscope, then extended into the ureter for a distance estimated by a surgeon.

Ureteral stents may also be inserted using fluoroscopy however, this procedure requires equipment that may not be available in all situations where a ureteral stent is being inserted and exposes the patient, and often the operator, to radiation.

Since prior art ureteral stents have no optical system, they are difficult to guide into position without the use of additional equipment. Further, they do not have the capability of performing inspection functions within the ureter.

### SUMMARY OF THE EMBODIMENTS

In a first aspect, an optically-guided ureteral stent includes an elongate body and an imager. The elongate body has a plurality of channels therethrough and a curve near a proximal end of the elongate body. The imager is disposed at the proximal end of the elongate body in a first channel of the plurality of channels. A second channel second channel of the plurality of channels has an opening at one of the proximal end and a lateral surface of the elongate body for passage of urine.

In another aspect, a method for inspecting a passageway with a ureteral stent includes advancing the ureteral stent in the passageway while capturing images with the imager at a proximal end of the ureteral stent. The captured images include a feature that is one of (i) an obstruction in the passageway and (ii) a branching of the passageway defined at least in part by an end of a desired branch adjoining the passageway. When the feature is the obstruction, the method includes steering the proximal end around the obstruction. When the feature is the branching, the method includes steering the proximal end into the desired branch.

### BRIEF DESCRIPTION OF THE FIGURES

FIG. 1 is a view of a curved tip of an optically-guided ureteral stent, in embodiments.

FIG. 2 is a side view of the curved tip of a ureteral stent of FIG. 1 showing the curved shape the elastomeric tip has when relaxed.

FIG. 3 is a tip view of the ureteral stent of FIG. 1 when a straightening rod is inserted into a channel of the tip of the stent.

FIG. 4 is a side view of the ureteral stent of FIG. 3 showing the straight shape of the elastomeric tip when the straightening rod is present at the tip of the stent.

FIG. 5 is an isometric view of the curved tip of the ureteral stent when the straightening rod is retracted from the stent tip.

FIG. 6 is an isometric view of the curved tip of the ureteral stent when the straightening rod is present at the stent tip.

FIG. 7 is an isometric view of the curved tip of the ureteral stent when the straightening rod is advanced beyond the stent tip.

FIG. 8 is a schematic the ureteral stent of FIG. 1 fitted with a handle, in embodiments.

FIG. 9 is a of a handle, which is an example of the handle of FIG. 8.

FIG. 10 is a schematic of an elongate body of a stent and a pusher that engages and rotates the elongate body, in embodiments.

FIGS. 11 and 12 are cross-sectional views of end regions of respective ureteral stents, in embodiments.

FIG. 13 includes isometric views and a cut-away view of an end region of a ureteral stent, in embodiments.

FIG. 14 is a flowchart illustrating a method for inspecting a passageway with a ureteral stent, in embodiments.

### DETAILED DESCRIPTION OF THE EMBODIMENTS

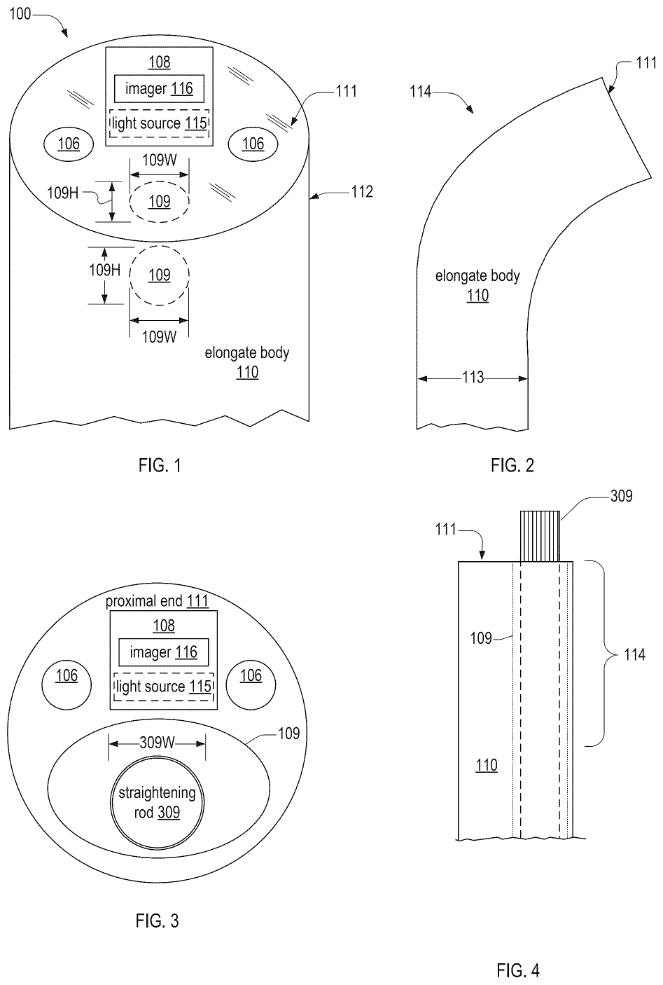

FIG. 1 shows an optically-guided ureteral stent 100. FIGS. 2 and 4 are side views of the curved tip of a ureteral stent of FIG. 1. FIG. 3 is a tip view of the ureteral stent of FIG. 1. FIGS. 1-4 are best viewed together in the following discussion.

A ureteral stent 100 as shown in FIG. 1 may be formed of an elastomeric material. In representative embodiments, optically-guided ureteral stent 100 has diameter of less than three millimeters but in other embodiments any diameter capable of fitting inside a body passageway may be used. A body passageway may include at least one of a person's urethra, bladder, and a ureter.

In embodiments, optically-guided ureteral stent 100 includes an elongate body 110. Elongate body 110 has a proximal end 111, a lateral surface 112, and a width 113, which may be a diameter. Width 113 may exceed one millimeter and may be less than three millimeters. Elongate body 110 may be formed by extrusion, and have a material composition that includes a polymer, such as a biocompatible silicone and polyurethane.

Elongate body 110 multiple channels extending therethrough. These include an illumination channel 106, a communication channel 108, and an drainage channel 109. Each channel has a width, such as a diameter, which may be between 0.3 mm and 2.5 mm.

An imager 116 is positioned at proximal end 111 for use when guiding ureteral stent 100 inside a body passageway. At least part of imager 116 may be within communication channel 108. Elongate body may also include a light source 115, which may be a light emitting diode. Light source 115 may be in communication channel 108 as shown in FIGS. 1 and 3, or in a different channel of elongate body 110.

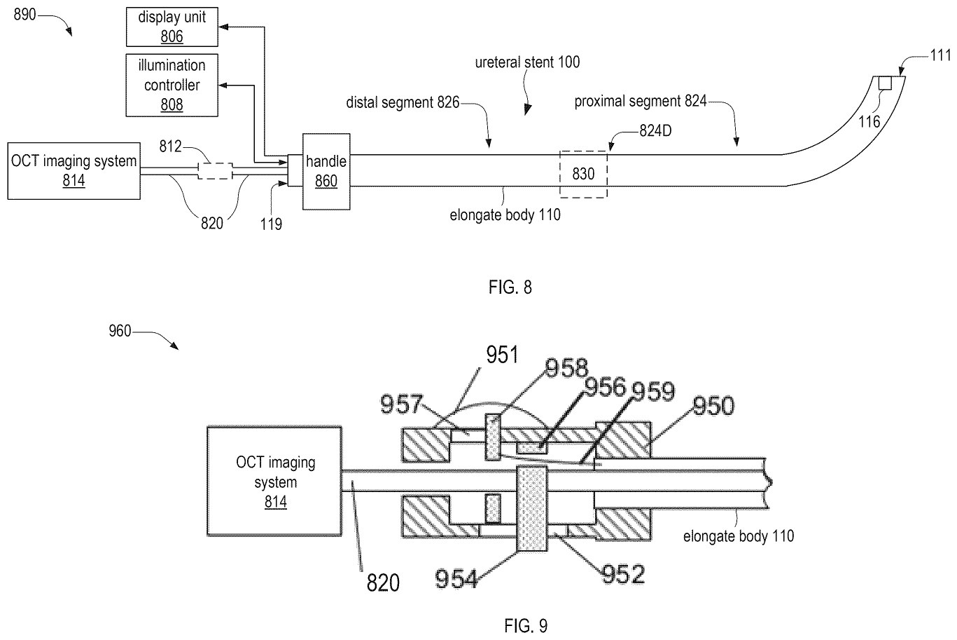

Communication channel 108 extends through elongate body 110 to carry electrical and/or optical fiber cables, which may couple imager 116 to a display unit 806 (FIG. 8) to provide images to an operator. Drainage channel 109 accommodate the passage of urine through ureteral stent 100, and may terminate at either proximal end 111. In embodiments, drainage channel 109 may terminate at lateral surface 112 near proximal end 111, to allow more area for imager 116 on proximal end 111.

Drainage channel 109 has a width 109W and a height 109H, at least one of which may be non-uniform along channel 109. For example, at proximal end 111, drainage channel 109 may be narrower in at least one dimension than further inside drainage channel 109 such that a stiffening wire may pass almost completely through drainage channel 109 while not protruding through proximal end 111 or lateral surface 112.

Elongate body 110 may also include one or more channels 106 extending through elongate body 110 for providing illumination in the vicinity of proximal end 111. Illuminators may include optical fibers to conduct light from external light sources to the proximal end of elongate body 110 or wiring to light source 115 disposed at or near the proximal end of elongate body 110. Channels 106 and 109 may be separate as shown and discussed herein, or may be combined in a single channel.

In some embodiments, wiring to light emitting diodes shares the same channel as the electrical and/or optical fiber cables coupling the imager 116 to display unit 806, and in some embodiments the light emitting diodes are formed as part of imager 116.

Imager 116 may be a commercially available camera module, which may include an image sensor and a lens aligned thereto. The camera module may also include at least one of an analog-to-digital converter and image-processor. The camera module dimensions may be less than or equal to 1 mm×1 mm×2 mm, with the final dimension being parallel to the optical axis of the camera module's lens. This permits placement of electronic cameras directly at proximal end 111 of ureteral stent 100 without need for coherent fiber bundles. It is expected future models may be even smaller and may include illuminator light-emitting diodes (LEDs) within the camera module to provide illumination. Small-diameter coherent fiber bundles are also available with diameters of about 0.4 mm and 4000 fibers, giving approximately 63×63 resolution; larger diameter bundles support higher resolutions. In an alternative embodiment, image sensing using a lens and coherent fiber bundle may be used.

When elongate body 110 includes light source 115, elongate body 110 may also include wires to provide power to light source 115. These wires extend through elongate body 110, and may be collocated with the electrical and/or optical fiber cables that couple imager 116 to display unit 806. Ureteral stent 100 may include traction thread in elongate body 110 that house wires connected to light source 115 and/or imager 116.

Drainage channel 109 may also be used with a straightening rod 309, which has a width 309W, which may be a maximum cross-sectional width of straightening rod 309. In embodiments, straightening rod 309 may also be referred to as a straightening wire. Straightening rod 309 may be a solid, elongated shape sized to move freely within drainage channel 109. In embodiments, straightening rod 309 may include a channel that provides for the delivery of fluids or lubricants to proximal end 111 of ureteral stent 100. Further, straightening rod 300 may be an OCT probe, an ultrasound probe or any type of probe capable of collecting images or other information about a body passage. References to straightening rod 309 should be understood as encompassing any of the embodiments discussed herein.

As shown in the side view of FIG. 2, ureteral stent 100 of FIG. 1 is formed with the tip of ureteral stent 100 having a molded curved shape. Curved tip 114 is flexible to accommodate passage through the body but retains this curved shape when there are no additional parts or surrounding tissue that would cause it to straighten The curved tip 114 as shown in FIG. 2 is referred to as the relaxed state of elongate body 110. In embodiments, curved tip 114 bends at a 70-degree angle when relaxed and no straightening rod or OCT probe (see below) is present. In an embodiment, curved tip 114 has a radius of curvature of four to ten and five millimeters, and the bend is directly adjacent to proximal end 111 of elongate body 110. In alternative embodiments, curved tip 114 bends between sixty degrees and a full 360 degrees with radius of curvature from three to ten millimeters. A straight distal segment of elongate body 110 may be between zero and fifteen millimeters long.

Drainage channel 109 is typically less than a millimeter in diameter, although it is large relative to other channels of stent 100. In larger-diameter ureteral stents of up to three millimeters diameter the drainage channel 109 may have larger diameters but, given space required for the imager 116, is typically less than two millimeters in diameter.

Optical coherence tomography (OCT) is based on low-coherence interferometry, typically employing near-infrared light. OCT may be used to identify changes in tissue resulting from thermal damage or other types of trauma. These changes may be detected on both the interior of a ureter or other body passageway, but also into and through the ureter wall. An OCT channel of a medical instrument requires one or more optical fibers to pass light to and from tissue of interest and to and from an OCT system and is known to provide useful diagnostic information from tissue in close proximity to a tissue end of the one or more optical fibers. To perform OCT imaging, low-coherent illumination light is provided to tissue and interference patterns returned from tissue are examined to provide OCT images.

As illustrated in FIGS. 3 and 4, a straightening rod 309 may be inserted in drainage channel 109. Straightening rod 309, which flexible to accommodate passage through the body, is stiffer than elongate body 110 so that it can cause curved tip 114 to straighten as straightening rod 309 is advanced to or beyond proximal end 111.

When straightening rod 309 is an OCT probe, it may have a single fiber, or multiple fibers, as appropriate to provide OCT imaging of tissue that may be encountered while navigating the ureteral stent through body passageways.

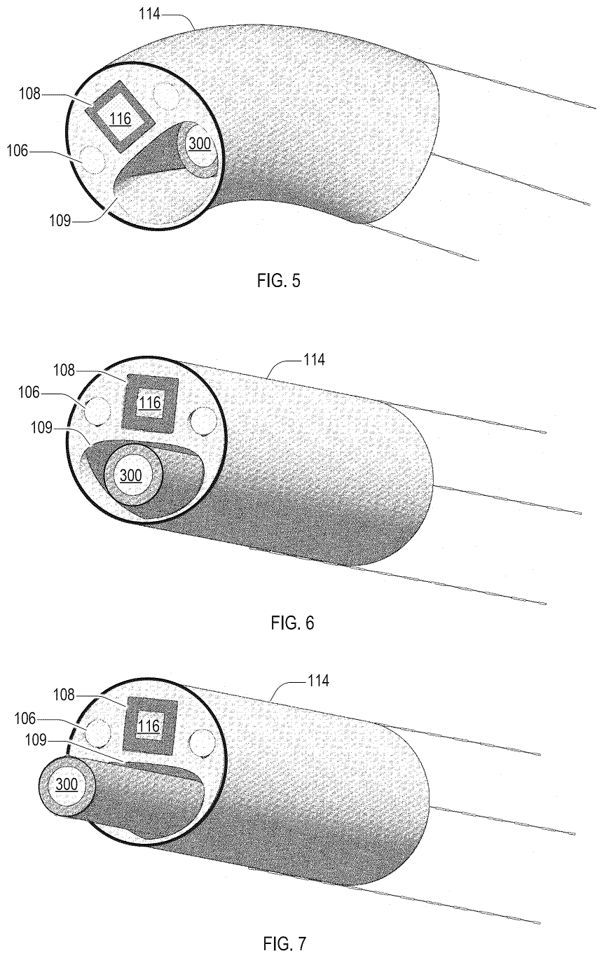

When ureteral stent 100 is being threaded through body passages that fork or branch, it is often necessary to manipulate the device into a particular branch so that it follows a desired pathway through the body passageways. It may also be necessary to steer ureteral stent 100 around obstructions in a passageway. Manipulation of ureteral stent 100 using straightening rod 309 will be described in connection with FIGS. 5-7, which are best viewed together in the following discussion.

As straightening rod 309 is advanced in elongate body 110, curved tip 114 retains its curved shape while straightening rod 309 is recessed away from proximal end 111, as shown in FIG. 5. Curved tip 114 is shown in the relaxed position. The distance between the proximal end of straightening rod 309 and proximal end 111 of elongate body 110 at which curved tip 114 retains its curved shape depends on the degree of bend of curved tip 114 and the relative stiffness of straightening rod 309 and elongate body 110.

In FIG. 6, straightening rod 309 has advanced to a position generally even with proximal end 111 so that curved tip 114 has become straight. This position is referred to as the neutral position of curved tip 114.

In FIG. 7, straightening rod 309 may be extended past proximal end 111 through the open end of drainage channel 109. This is referred to as an extended position and may be used when straightening rod 309 is an OCT or ultrasound probe for collecting information about the body passage surrounding ureteral stent 100.

When it is desired to steer ureteral stent 100 as herein described, straightening rod 309 is inserted, or withdrawn, through drainage channel 109 to straighten curved tip 114 near proximal end 111 to straighten the curved portion (in first position) or bend (in second position) curved tip 114 to an appropriate angle, and ureteral stent 100 is rotated to aim proximal end 111 into the desired branch of the body passageway as the ureteral stent is advanced into the desired branch. The curved portion near proximal end 111 may take on any desired angle between zero degrees (straight) and the approximately seventy degrees, which, in embodiments, is the angular orientation of the relaxed position of curved tip 114.

While ureteral stent 100 is being advanced through the ureter, any suspicious tissue found may be inspected by imaging with imager 116 and/or an OCT or ultrasound probe forming straightening rod 309. Upon reaching the renal pelvis through the ureter, ureteral stent 100 may be navigated into any desired major or minor calyx to inspect any suspect tissue, such as possible tumor tissue identified on preoperative imaging.

When ureteral stent 100 is used to stent the ureter, straightening rod 309 may be withdrawn, and elongate body 110 left in place as long as necessary. Curved tip 114 of elongate body 110 helps anchor elongate body 110 in place until healing takes place, and it is time to remove the stent.

FIG. 8 is a block diagram of a system embodying the ureteral stent 100. Ureteral stent has a handle 860. FIG. 9 is a cross sectional schematic diagram of a handle 960 at the distal end of ureteral stent 100. Handle 960 is an example of handle 860. FIGS. 8 and 9 are best viewed together in the following description.

FIG. 8 is a representative block diagram of a system 890 incorporating a ureteral stent 100. Elongate body 110 may be fitted with handle 860 near its distal end 119. Handle 860 can manipulate elongate body 110, for example, by being rotated or advanced into or through a channel of a body passage to be inspected.

Imager 116 at proximal end 111 of elongate body 110 is coupled to an image display unit 806 that may include a recording system. Illumination optical fibers or wires to LEDs passing through third channel(s) 106 (FIGS. 1 and 2) and or channel 108 of elongate body 110 may be coupled to an illumination controller 808. An illumination optical fiber may be coupled to a laser used for laser lithotripsy.

In embodiments, system 890 includes at least one of a second handle 812 and a straightening rod 820. Straightening rod 820 may incorporate an imaging probe such as an OCT imaging probe or ultrasound imaging probe. Straightening rod 820 may extends through second handle 812. Second handle 812 can be used to manipulate straightening rod 820 in ways including at least one of (i) extending the imaging probe or other straightening rod 820 into or through drainage channel 109, (ii) withdrawing the imaging probe or other straightening rod fully or in part from drainage channel 109.

When straightening rod 820 includes an OCT probe, straightening rod 820 may be part of, or be communicatively coupled to, an OCT imaging system 814, which provides low-coherence illumination and examine interference patterns returned through optical fibers of OCT probe as straightening rod 820 and provide OCT images therefrom. If a simple straightening rod 820 is used in place of OCT probe, second handle 812 is present but OCT imaging system 814 is omitted.

### Segmented Elongate Body

In embodiments, elongate body 110 includes a proximal segment 824 and a distal segment 826. In such embodiments, elongate body 110 is part of a ureteral stent where, of segments 824 and 826, only proximal segment 824 of elongate body 110 remains in a patient to pass urine from kidney to bladder.

In such embodiments, elongate body 110 includes a connector 830. Connector 830 removably attaches segments 824 and 826 while also aligning corresponding channels of segments 824 and 826 such that, for example, at least one of fluid, an electrical wire, and an optical fiber traverse connector 830 from between segments 824 and 826 uninterrupted. Connector 830 may itself have channels that align with channels of segments 824 and 826, such that segments 824 and 826 are not abutting when connected.

Connector 830 may be an remotely actuatable connector, such that handle 860 controls connector 830, e.g., a latch thereof, to disconnect distal segment 826 from proximal segment 824. Connector 830 include a mechanism of remote disconnection may be electrical, mechanical, electro-magnetic (e.g., via an electromagnet), and any combination thereof.

A proximal part of connector 830 may be attached or integrated into proximal segment 824; and distal part of connector 830 may be attached or integrated into distal segment 826. Upon disconnection, at least part of connector 830 may remain attached to either or both of proximal segment 824 and distal segment 826.

Connector 830 is positioned between proximal end 111 and handle 860 at a point such that, when ureteral stent 100 is separated at connector 830, proximal segment 824 may remain in a patient's ureter to serve as a ureteral stent while distal segment 826 is withdrawn through the urethra. Stent 100 may remain in the ureter for a period of time required for the patient's ureter to properly heal. This period of time may be one week, two weeks, or longer.

A distal end 824D of proximal segment 824 may remain in the bladder until, for example, it is seized by a removal tool extended through a cystoscope, whereupon proximal segment 824 can be removed from the ureter and bladder, and drawn out through the patient's urethra.

When elongate body 110 includes connector 830 and imager 116 is electrically connected to display unit 806, connector 830 may function as both a mechanical connector and an electrical connector, such as a zero-insertion-force connector. That is, connector 830 may electrically connect imager 116 to display unit 806 and/or electrically connect light source 115 to illumination controller 808. In one embodiment, connector 830 provides secure electrical contact between electrical leads of proximal segment 824 to distal segment 826. This electrical contact may be released by actuating connector 830, e.g., by pulling an actuating wire located in an additional channel of distal segment 826. The electrical leads proximal segment 824 may be connected at least one of imager 116 and light source 115.

Various embodiments of connector 830 are anticipated. In one embodiment of connector 830, each of segments 824 and 826 have channels that align with each other, where one of segments 824 has a conic protrusion that enters a conic depression in the other of segments 826 to help maintain alignment. In embodiments, electrical connections of segments 824 and 826 each terminate in a lengthwise row of aligned contacts alongside the channels and a wedge is inserted past a detent to hold the contacts together. The wedge may be coupled to the release wire such that pulling the release wire allows the contacts to separate to permit disengagement of the coupler/connector. In another embodiment of connector 830, a clip attached to the release wire clamps together a protrusion on each of segments 824 and 826, pulling the release wire releases the protrusions and allows the distal and proximal segments to separate, thereby separating aligned electrical contacts of segments 824 and 826.

In alternative embodiments, second handle 812 is mounted on handle 860 and configured as a slider configured for thumb. This permits one-hand operation of the ureteral stent 100 using a hand to rotate and advance or withdraw the ureteral stent 100 with handle 860 gripped with the palm and fingers, while using the slider serving as second handle 812 to extend or retract the OCT port or straightening rod 820. In an alternative embodiment, OCT port or straightening rod 820 is replaced by a small-diameter ultrasound probe.

Straightening rod 820 may be or include an OCT or other imaging probe, and one of the channels of elongate body 110 houses N optical fibers, where N is greater than or equal to one. The N optical fibers couple light from external OCT imaging system 814 to tissue located either at an end of the optical fiber, or via a mirror to a point lateral to the N optical fibers. Scattered and reflected light propagates from the tissue back to the OCT imaging system. In an alternative embodiment, the OCT probe or straightening rod 820 is replaced with a small diameter ultrasound imaging probe and OCT imaging system 814 is replaced by an ultrasound imaging system.

FIG. 9 is a schematic of a handle 960 in a use scenario where it is controlling elongate body 110. Handle 960 is an example of handle 860, FIG. 8. Handle 960 includes a body 950 that has a slot 952. through which a portion of a thumb-slider 954 of a device 956 attached to the OCT port fibers or straightening rod 820 that serves as OCT port or straightening rod handle 812. In a particular embodiment, body 950 also includes a slot 957. Through slot 957 is a portion of a second thumb slider 958, which is coupled to an actuating wire 959. Actuating wire 959 is configured to release connector 830. Second thumb slider 958 may be covered by a protective cover 951 such that two motions are required to release connector 830: a first motion to remove protective cover 951, and a second motion to pull second thumb slider 958.

In embodiments used as ureteral stents, a diameter of elongate body 110 and proximal end 111 may be between approximately one and three millimeters. A diameter of elongate body 110 and proximal end 111 may be less than approximately 0.1 and 0.2 millimeters and channel diameter may be between approximately 0.3 and one millimeter.

In some embodiments, elongate body 110 is formed by extruding a soft thermoplastic core having a channel, or channel, for connecting wires and/or optical fibers associated with the imager 116. The core is placed in a mold having a tip bend and heated to set a bend near a distal tip of the core. The imager 116 is then attached the distal tip of the core and its lead wires and/or optical fibers are inserted in the channel. The core may be formed with drainage channel 109 integral to the core.

The core is then dipped into a flexible, biocompatible, coating to seal the channel and the coating is cured. In an alternative embodiment a thin, biocompatible heat-shrink tubing is placed over the core and channel and shrunk onto the core, the channel becoming a channel with core on some sides of the channel and an inner surface of the heat-shrink tubing on another side of the channel; multiple channels may be formed in the body in this way. In an alternative embodiment, drainage channel 109 is also formed by extruding a channel when the core is extruded, and closing the channel with the heat-shrink tubing.

### Rotating a Stent About its Axis for Steering

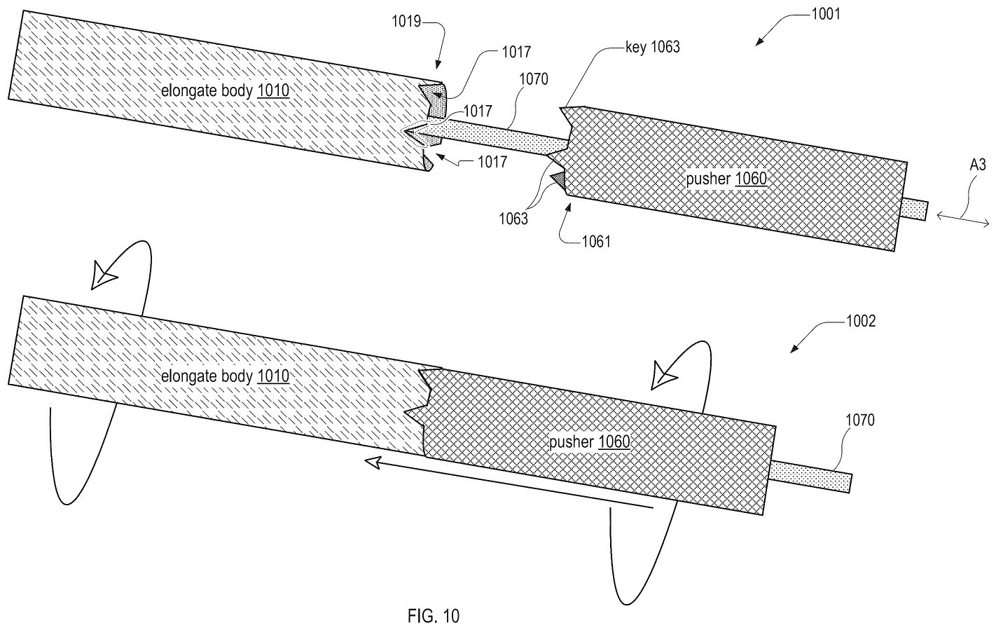

Accurate steering a ureteral stent 100 benefits from the ability to rotate the stent about its axis. FIG. 10 includes schematics 1001 and 1002, which include an elongate body 1010 of a stent and a pusher 1060. Elongate body 1010 is an example of elongate body 110. Pusher 1060 is an example of handle 860, FIG. 8, or may be part of handle 860.

In schematics 1001 and 1002, a guidewire 1070 is within each of elongate body 1010 and pusher 1060. Guidewire 1070 is parallel to an axis A3 about which elongate body 1010 and pusher 1060 may be concentric. Axis A3 is parallel to a longitudinal axis of a straight section of elongate body 1010. In schematic 1001, elongate body 1010 and pusher 1060 are spatially separated. In schematic 1002, pusher 1060 is mechanically engages with elongate body 1010, and hence rotates elongate body 1010 when pusher 1060 is rotated about axis A3.

Elongate body 1010 has a distal end 1019; pusher 1060 has a proximal end 1061. Distal end 1019 and proximal end 1061 are mechanically keyed to allow pusher 1060 to engage and rotate elongate body 1010. Elongate body 1010 has one or more keys 1017 at its distal end 1019. Pusher 1060 has one or more keys 1063 at its proximal end 1061. In embodiments, each key 1017 is convex (“male”) and each key 1063 is concave (“female”). In other embodiments, each key 1017 is concave and each key 1063 is convex. Pusher 1060 may be rotationally oriented about axis A3 such that each key 1063 engages with a respective key 1017. Keys 1017 and 1063 may be positioned such that more than one rotational orientation of pusher 1060 results in each key 1063 being aligned with, and hence engageable to, with a respective key 1017.

### Camera Module Accommodation in Stent Channel

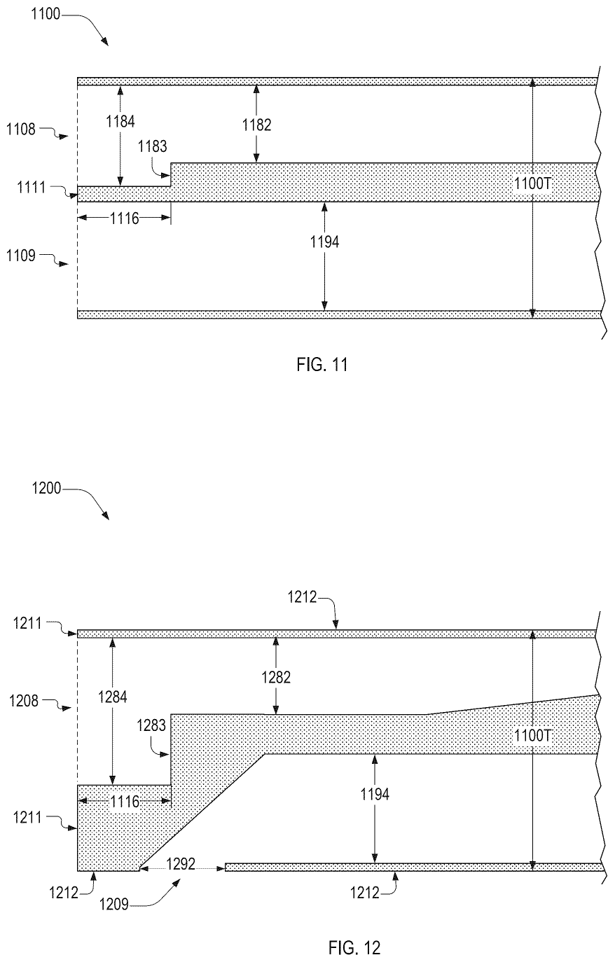

FIG. 11 is a cross-sectional view of a end region 1100 of a ureteral stent. End region 1100 may be the end region of an elongate body, such as elongate body 110. Alternatively, end region 1100 may be a ferrule that attaches to elongate body 110. The ferrule's material composition may include a biocompatible resin.

End region 1100 includes a communication channel 1108 and an drainage channel 1109, which are respective examples of communication channel 108 and drainage channel 109. End region 1100 has a proximal end 1111. At proximal end 1111, channels 1108 and 1109 have respective heights 1184 and 1194, and end region 1100 has a thickness 1100T. Further within channel 1108, at a distance 1116 from distal end 1111, channel 1108 has an interior height 1182, which is less than 1184. At distance 1116, channel 1108 includes a vertical wall 1183. Distance 1116 may be between one millimeters and three millimeters.

When channels 1108 and 1109 accommodate imager 116 and straightening rod 309, respectively, heights 1184 and 1194 must exceed respective heights of imager 116 straightening rod 309, while also being less than thickness 1100T. Imager 116 has a height that is between heights 1182 and 1184 such that the location of vertical wall 1183 determines a maximum depth of imager 116 within channel 1108. Channel 1108 may also accommodate one or more optical fibers used for illumination.

FIG. 12 is a cross-sectional view of a end region 1200 of a ureteral stent. End region 1200 may be the end region of an elongate body, such as elongate body 110. Alternatively, end region 1200 may be a ferrule that attaches to elongate body 110. The ferrule's material composition may include a biocompatible resin. End region 1200 has thickness equal to thickness 1100T of end region 1200.

End region 1200 includes a channel 1208 and a channel 1209, which are respective examples of communication channel 108 and drainage channel 109. End region 1100 has a proximal end 1211 and a lateral surface 1212. Whereas channel 1109 of end region 1100 terminates at distal end 1111, channel 1209 terminates at lateral surface 1212. At proximal end 1211, channel 1208 has a height 1284. Further within channel 1208, at a distance 1116 from distal end 1211, channel 1108 has an interior height 1282, which is less than height 1294. At distance 1116, channel 1208 includes a vertical wall 1283, which has the same function of vertical wall 1183.

The side-termination of drainage channel 1209 enables height 1284 channel 1208 to exceed with 1184 of channel 1108 of end region 1100, FIG. 11, and hence also enables a stent 100 with end region 1200 to accommodate a larger imager 116 than does a stent 100 with end region 1100. At lateral surface 1212, channel 1209 has a width 1292.

In embodiments, width 309W of straightening rod 309 is less than width 1194 and exceeds width 1292, such that straightening rod 309 can extend into end region 1200 while not protruding from proximal end 1211. Width 1292 may exceed that of guidewire 1070, such that guidewire 1070 may extend entirely through channel 1209, which allows for stent 100 to be exchanged for a new ureteral stent with guidewire 1070 in place within a patient. For example, width 1292 may exceed 0.9 millimeters such that both guidewire 1070 and fluid may enter and exit drainage channel 1209.

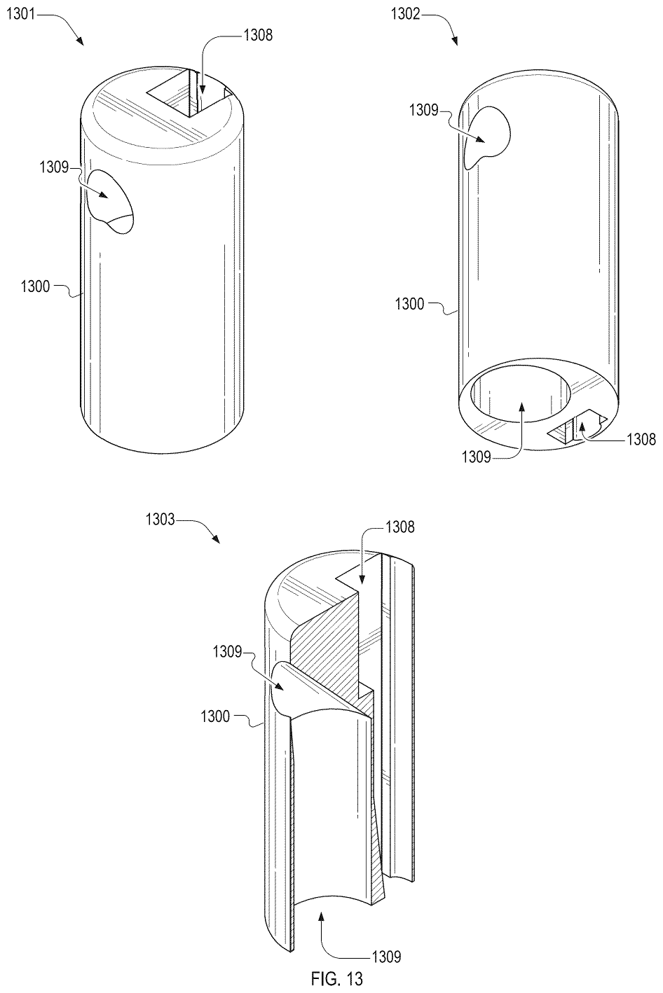

FIG. 13 includes isometric views 1301 and 1302 and a cut-away view 1303 of an end region 1300 of a ureteral stent, in embodiments. End region 1300 is an example of end region 1200. End region 1300 includes a channel 1308, channel 1309, a proximal end 1311, and a lateral surface 1312, which are respective examples of channel 1208, channel 1209, proximal end 1211, and lateral surface 1212 of end region 1200.

### Method of Inserting and Navigating a Ureteral Stent

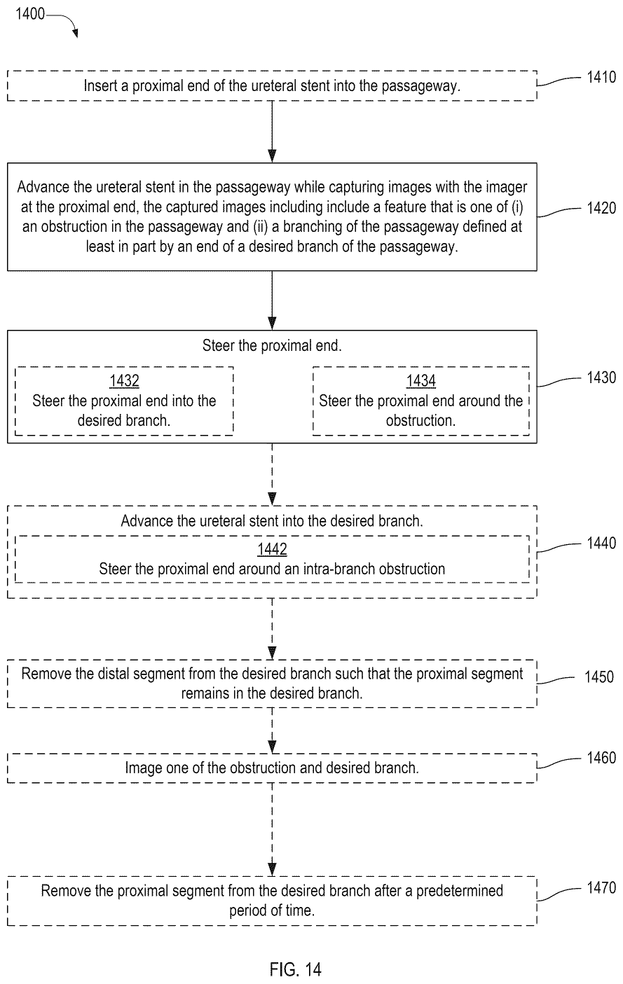

FIG. 14 is a flowchart of an example method for inspecting a passageway with a ureteral stent. The passageway may include the urethra, the bladder, a ureteric orifice, and the ureter of a patient. Method 1400 may be implemented using ureteral stent 100. Method 1400 includes steps 1420, and 1430. Method 1400 may also include at least one of steps 1410, 1440, 1450, 1460, and 1470. Step 1410 includes inserting the proximal end of the ureteral stent into the passageway.

Step 1420 includes advancing the ureteral stent in the passageway while capturing images with the imager at the proximal end. The captured images include a feature that is one of (i) an obstruction in the passageway and (ii) a branching of the passageway defined at least in part by an end of a desired branch adjoining the passageway. The obstruction may be a ureteral tumor, a kidney stone, or a kink in the ureter caused by a misplaced suture.

Step 1430 includes steering the proximal end. When the feature is the branching, step 1430 includes step 1432, which includes steering the proximal end into the desired branch. The desired branch may include a ureter of the patient, where the entrance to the branch is the ureteric orifice. When the feature is the obstruction, step 1430 may include step 1434, which includes steering the proximal end around the obstruction.

Step 1430 may include rotating the ureteral stent about a longitudinal axis of the ureteral stent. When the ureteral stent includes a channel and a straightening rod in the channel, step 1430 may include manipulating a straightening rod within a channel to flex the proximal end of the ureteral stent to an appropriate angle so the ureteral stent, or at least the proximal end, enters the desired branch or avoids the obstruction.

Step 1440 includes advancing the ureteral stent into the desired branch. In steps 1430 and 1440, an operator may use at least one of handle 860 and pusher 1060 to position and advance proximal end 111 of elongate body 110. The captured images may include an intra-branch obstruction in the desired branch, in which step 1440 may include step 1442. Step 1442 includes steering the proximal end around the intra-branch obstruction.

Step 1450 is applicable when the feature is the branching and the ureteral stent includes an elongate body having a distal segment and a proximal segment that includes the imager. Step 1450 includes removing the distal segment from the desired branch such that the proximal segment remains in the desired branch. Segments 824 and 826 are respective examples of the distal segment and the proximal segment.

Step 1460 includes imaging one of the obstruction and the desired branch. The imaging of 1460 may include imaging one of the obstruction and the desired branch via one or more of (i) optical coherence tomography imaging through an OCT probe of a straightening rod in a channel of the ureteral stent and (ii) an ultrasound imaging probe of a straightening rod in a channel of the ureteral stent.

When method 1400 includes step 1460 and the feature is the branching, method 1400 may also include step 1470. Step 1470 includes removing the proximal segment from the desired branch after a predetermined time period that exceeds one day. The time period may be less than one month, for example.

The ureteral stent herein described is also useful in other medical applications including inspecting other body passageways such as intestines or nasal passageways, or observing laparoscopic procedures from different or additional angles from those traditionally used, and in non-medical applications where it can be adapted for inspecting valves and cylinders of gasoline engines or hot sections of gas turbines as well as water and sewage pipes or air ducts.

Changes may be made in the above methods and systems without departing from the scope hereof. It should thus be noted that the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall therebetween.

Changes may be made in the above methods and systems without departing from the scope of the present embodiments. It should thus be noted that the matter contained in the above description or shown in the accompanying drawings should be interpreted as illustrative and not in a limiting sense. Herein, and unless otherwise indicated the phrase “in embodiments” is equivalent to the phrase “in certain embodiments,” and does not refer to all embodiments. The following claims are intended to cover all generic and specific features described herein, as well as all statements of the scope of the present method and system, which, as a matter of language, might be said to fall therebetween.

1. An optically-guided ureteral stent comprising,
an elongate body having a plurality of channels therethrough and a curve near a proximal end of the elongate body;
an imager disposed at the proximal end of the elongate body in a first channel of the plurality of channels; and
a second channel of the plurality of channels having an opening at one of the proximal end and a lateral surface of the elongate body for passage of urine.

2. The ureteral stent of claim 1, further comprising an illuminator disposed at the proximal end of the elongate body in a third channel of the plurality of channels.

3. The ureteral stent of claim 2, wherein the third channel further comprises a plurality of illuminator channels, each having a respective illuminator at the proximal end of the elongate body.

4. The ureteral stent of claim 1, further comprising:
a straightening rod disposed within the second channel of the elongate body;
a first handle coupled to the elongate body that manipulates the elongate body; and
a second handle coupled to the straightening rod that advances and retracts the straightening rod in the second channel of the elongate body.

5. The ureteral stent of claim 4, wherein the straightening rod includes an optical coherence tomography (OCT) probe.

6. The ureteral stent of claim 4, wherein the straightening rod further includes an ultrasound imaging probe.

7. The ureteral stent of claim 4, wherein the straightening rod further includes a channel for providing fluid to the proximal end of the elongate body.

8. (canceled)

9. The ureteral stent of claim 1, the elongate body comprising a proximal segment and a distal segment, and further comprising a connector coupling the proximal segment to the distal segment.

10. (canceled)

11. The ureteral stent of claim 1, wherein the imager comprises an electronic camera disposed at the proximal end of the elongate body, or a combination of lens disposed at the proximal end and a coherent fiber bundle extending through the first channel and an electronic camera.

12. The ureteral stent of claim 1, the opening of the drainage channel being at the lateral surface of the elongate body.

13. An optically-guided ureteral stent system comprising:
the ureteral stent of claim 1; and
display unit communicatively coupled to the imager.

14. (canceled)

15. A method for inspecting a passageway with a ureteral stent, comprising:
advancing the ureteral stent in the passageway while capturing images with the imager at a proximal end of the ureteral stent, the captured images including a feature that is one of (i) an obstruction in the passageway and (ii) a branching of the passageway defined at least in part by an end of a desired branch adjoining the passageway;
when the feature is the obstruction, steering the proximal end around the obstruction;
and
when the feature is the branching, steering the proximal end into the desired branch.

16. The method of claim 15, the feature being the branching, the passageway including an interior of a bladder of a patient, the branching being a ureteric orifice of the patient, and the desired branch being a ureter of the patient

17. The method of claim 15, steering comprising rotating the ureteral stent about a longitudinal axis of the ureteral stent.

18. The method of claim 15, the ureteral stent including a channel and a straightening rod in the channel, steering comprising manipulating a straightening rod within a channel to flex the proximal end of the ureteral stent to an appropriate angle so the proximal end enters the desired branch or avoids the obstruction.

19. The method of claim 15, the feature being the branching, and further comprising advancing the ureteral stent into the desired branch.

20. (canceled)

21. The method of claim 15, the feature being the branching, the ureteral stent including an elongate body having a distal segment and a proximal segment that includes the imager, and further comprising:
removing the distal segment from the desired branch such that the proximal segment remains in the desired branch.

22. (canceled)

23. (canceled)

24. The method of claim 15, further comprising imaging one of the obstruction and the desired branch via optical coherence tomography imaging through an OCT probe of a straightening rod in a channel of the ureteral stent.

25. The method of claim 15, further comprising imaging one of the obstruction and the desired branch via ultrasound imaging with an ultrasound imaging probe of a straightening rod in a channel of the ureteral stent.

26. The method of claim 15, further comprising inserting the proximal end of the ureteral stent into the passageway.
